(19) United States
(12) Patent Application Publication
(10) Pub. No.: US 2026/0216468 A1
(43) Pub. Date: Jul. 30, 2026

(54) LOCK FOR ENDOTRACHEAL TUBE VALVE

(71) Applicant: Cody Cronk, Pomona, CA (US)

(72) Inventor: Cody Cronk, Pomona, CA (US)

(21) Appl. No.: 19/457,441

(22) Filed: Jan. 23, 2026

Related U.S. Application Data
(60) Provisional application No. 63/749,834, filed on Jan. 27, 2025.

(51) Int. Cl. A61M 16/20; A61M 16/04; A61M 16/08

(52) U.S. Cl. CPC A61M 16/201; A61M 16/0402; A61M 16/0434; A61M 16/0816

(57) ABSTRACT

An endotracheal tube assembly and method comprises a tubing body and a valve including a valve body and a valve member. The valve member is rotatable within a valve chamber between an open position that allows fluid communication through the tubing body and a closed position that prevents fluid flow, thereby maintaining positive pressure during ventilator disconnections. A lock secures the valve member in the open position, preventing inadvertent closure. The lock includes a plate portion with a slot configured to receive a grip of the valve member and a pair of flexible arms extending along opposing sides of the valve body. The arms engage the valve body via a detent-and-groove arrangement, providing a snap-fit connection. The lock is selectively removable by applying an outward force that spreads the flexible arms, thereby moving the valve member to the closed position.

### CROSS-REFERENCE TO RELATED APPLICATIONS

This application claims the benefit of U.S. Provisional Patent Application No. 63/749,834, which was filed on Jan. 27, 2025, the contents of which are incorporated herein by reference in their entirety.

### BACKGROUND

Endotracheal tubes are essential devices used in medical practice to maintain an open airway in patients requiring mechanical ventilation or respiratory support. These tubes are often equipped with connectors or adapters to connect to mechanical ventilators and other respiratory devices. While the current designs and functions of endotracheal tubes have significantly advanced patient care, several challenges and limitations remain, particularly in specific clinical scenarios.

One critical concern involves patients requiring high levels of peak end-expiratory pressure (PEEP), such as those suffering from acute respiratory distress syndrome (ARDS). PEEP is vital for maintaining alveolar recruitment and optimizing oxygenation by preventing alveolar collapse during the expiratory phase of ventilation. However, when a mechanical ventilator is connected or disconnected from an endotracheal tube inserted into a patient's airway, there is often a transient loss of positive pressure. This loss can lead to alveolar derecruitment, adversely affecting the patient's respiratory status and overall stability.

This issue is not limited to ARDS patients; it also affects individuals at risk of alveolar derecruitment due to other conditions or factors. For these patients, connecting or disconnecting the mechanical ventilator poses significant challenges for maintaining stable airway pressures and preventing complications.

To address these concerns, it has been proposed to incorporate valves into endotracheal tubes. These valves are designed to block fluid flow through the tube selectively, thereby preventing loss of positive pressure during ventilator disconnections. However, these valves may present their own challenges, particularly the risk of inadvertent closure during use, which could compromise airway patency and patient safety. As such, there is a need for a mechanism that reliably holds the valve open during operation while allowing intentional closure when required.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale, or in schematic form in the interest of clarity and conciseness. Not every component may be labeled in every drawing. Like reference numerals in the figures may represent and refer to the same or similar element or function. In the drawings:

FIG. 1 is a perspective view of an exemplary endotracheal tube assembly constructed in accordance with the inventive concepts disclosed herein, showing a lock securing a tubing adapter in an open position.

FIG. 2 is a perspective view of the tubing adapter of the endotracheal tube assembly shown in FIG. 1.

FIG. 3A is a perspective view of an exemplary valve body of the tubing adapter shown in FIG. 2.

FIG. 3B is a rear elevational view of the valve body of FIG. 3A.

FIG. 3C is a front elevational view of the valve body of FIG. 3A.

FIG. 4A is a cross-sectional view of the valve body taken along line 4A-4A of FIG. 3A.

FIG. 4B is a cross-sectional view of another embodiment of a valve body.

FIG. 5 is a perspective view of an exemplary valve member of the tubing adapter shown in FIG. 2.

FIG. 6 is a side elevational view of the valve member shown in FIG. 5.

FIG. 7 is a cross-sectional view of the valve member taken along line 7-7 of FIG. 5.

FIG. 8A is a cross-sectional view of the tubing adapter taken along line 8A-8A of FIG. 2, wherein the valve member is shown in an open position.

FIG. 8B is a cross-sectional view of the tubing adapter shown in FIG. 2, wherein the valve member is shown in a closed position.

FIG. 9 is a perspective view of the tubing adapter of the endotracheal tube assembly shown in FIG. 1 with a lock attached thereto.

FIG. 10 is a perspective view of the lock.

FIG. 11 is a cross-sectional view of the lock taken along line 11-11 of FIG. 10.

FIG. 12 is a side elevational view of the tubing adapter and the lock with the lock engaged with the tubing adapter.

FIG. 13 is a side elevational view of the tubing adapter and the lock, with the lock shown disengaged from the tubing adapter.

### DETAILED DESCRIPTION OF EXEMPLARY EMBODIMENTS

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein are for the purpose of description and should not be regarded as limiting.

Headings are provided for convenience only and are not to be construed to limit the invention in any manner. Embodiments illustrated under any heading or in any portion of the disclosure may be combined with embodiments illustrated under the same or any other heading or portion of the disclosure. Any combination of the elements described herein in all possible variations thereof is encompassed by the invention unless otherwise indicated herein or otherwise clearly contradicted by context.

Unless otherwise required by context, singular terms shall include pluralities, and plural terms shall include the singular, except that the term “plurality,” as used herein, does not include the singular.

All patents or published patent applications referenced in any portion of this application are herein expressly incorporated by reference in their entirety to the same extent as if each individual patent or publication were specifically and individually indicated to be incorporated by reference.

All assemblies, systems, kits, and/or methods disclosed herein can be made and executed without undue experimentation in light of the present disclosure. Where a method claim does not explicitly state in the claims or description that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of embodiments described in the specification.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” The term “plurality” refers to “two or more.”

The use of the term “at least one” will be understood to include one as well as any quantity of more than one. In addition, the use of the term “at least one of X, Y, and Z” will be understood to include X alone, Y alone, and Z alone, as well as any combination of X, Y, and Z.

The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The term “or” in the claims means an inclusive “and/or” unless explicitly indicated to refer to alternatives only or unless the alternatives are mutually exclusive.

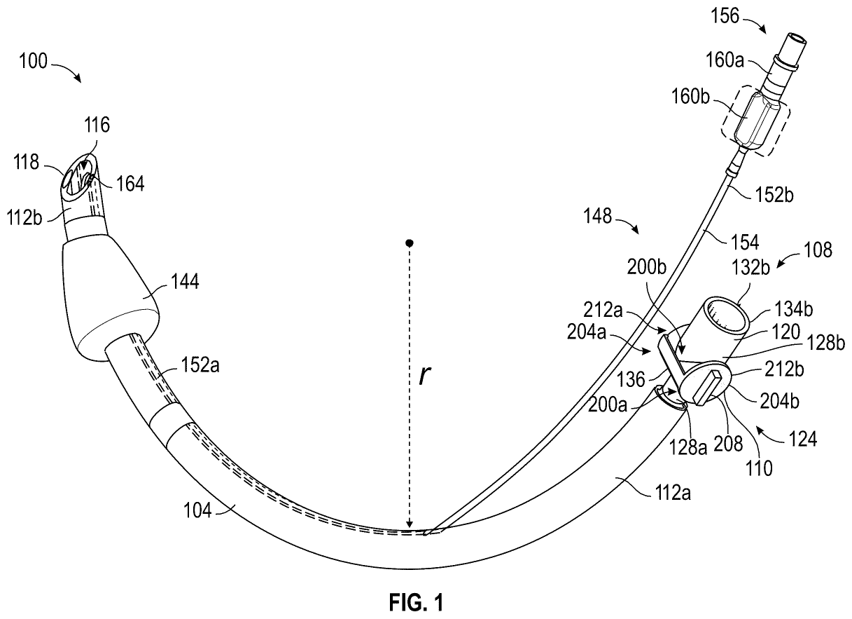

Referring now to the drawings, and in particular to FIG. 1, shown therein is a perspective view of an exemplary endotracheal tube assembly 100 (hereinafter the “assembly 100”) constructed in accordance with the inventive concepts disclosed herein. The assembly 100 may comprise a tubing body 104 and a valve or valve 108. The valve 108 is secured in an open position with a lock 110.

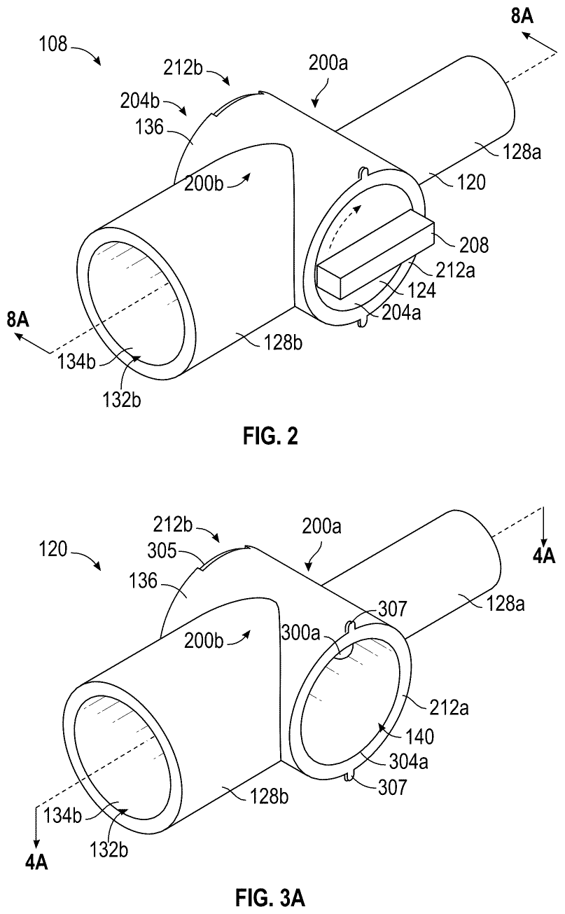

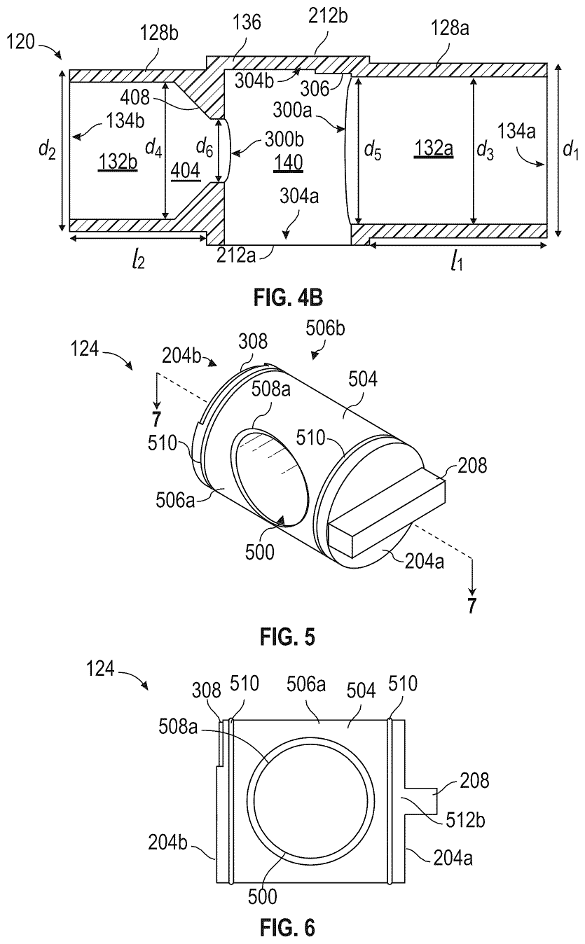

Referring now to FIG. 2, a perspective view of the valve 108 is shown. The valve 108 includes a valve body 120 and a valve member 124. The valve body 120 has a first connector portion 128a defining a first connector flow passage 132a (shown in FIG. 4A) having a first opening 134a (shown in FIG. 4A), and a second connector portion 128b defining a second connector flow passage 132b having a second opening 134b. The first connector portion 128a is matingly engaged with the proximal end 112a of the tubing body 104, so the valve body 120 extends outwardly from the proximal end 112a of the tubing body 104, and the first connector flow passage 132a is in fluid communication with the lumen 116 of the tubing body 104.

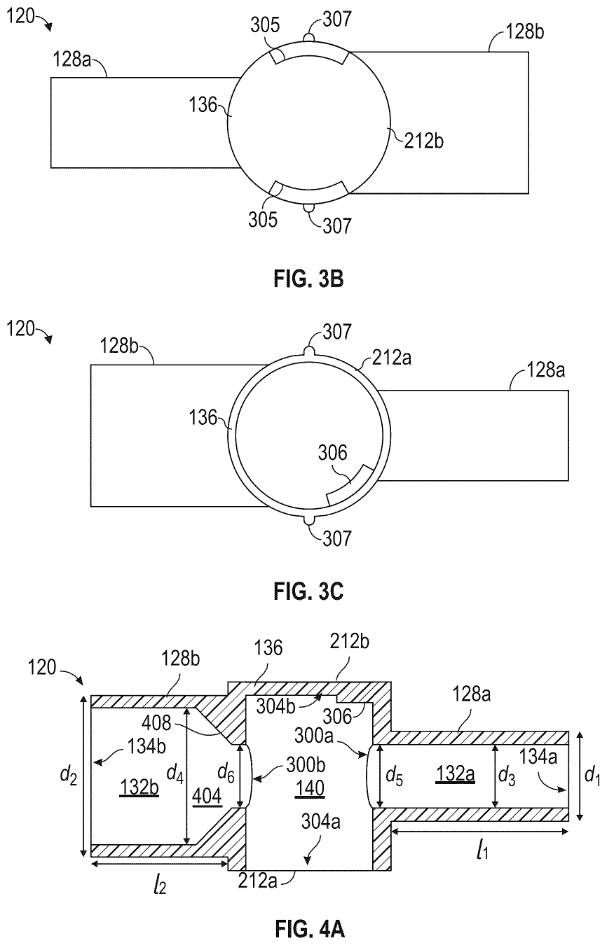

The valve body 120 has a valve portion 136 disposed between the first connector portion 128a and the second connector portion 128b. The valve portion 136 defines a valve chamber 140 (shown in FIGS. 3A and 4A), wherein the valve member 124 is positioned in the valve chamber 140 and rotatable between a first position (i.e., an open position), as shown in FIG. 1, and a second position (i.e., a closed position) (shown in FIG. 8B).

As shown in FIG. 2, the valve portion 136 of the valve body 120 has a first side 200a and a second side 200b opposite the first side 200a. The first connector portion 128a of the valve body 120 extends outwardly from the first side 200a of the valve portion 136, and the second connector portion 128b extends outwardly from the second side 200b of the valve portion 136.

In some implementations, as shown in FIG. 2, the first connector portion 128a, the second connector portion 128b, and the valve portion 136 each have a cylindrical shape; however, in other implementations, one or more of the first connector portion 128a, the second connector portion 128b, and the valve portion 136 may have a shape other than a cylindrical shape, such as a prismatic shape, for example.

The valve member 124 has a first end 204a and a second end 204b opposite the first end 204a. Similarly, the valve portion 136 of the valve body 120 has a first side 212a and a second side 212b opposite the first side 212a.

The valve member 124 may have one or more handles or grips 208 (hereinafter “grip 208”) extending outwardly from one or more of the first end 204a and the second end 204b. While the valve member 124 shown in FIG. 2 has one grip 208, other implementations may have more or fewer than two grips 208. By grasping the grip 208, a user may rotate the valve member 124 in the valve chamber 140 (shown in FIG. 3A) to move the valve member 124 between the first position (i.e., the open position), as shown in FIG. 2, and the second position (i.e., the closed position) (shown in FIG. 8B).

Referring now to FIG. 3A, shown therein is a perspective view of the valve body 120 of the valve 108 shown in FIG. 2. As shown in FIG. 3, the valve chamber 140 defined by the valve portion 136 may extend between the first side 212a of the valve portion 136 and the second side 212b of the valve portion 136. The valve portion 136 defines a first opening 300a in fluid communication with the first connector flow passage 132a and a second opening 300b (shown in FIG. 4A) in fluid communication with the second connector flow passage 132b. Further, the valve portion 136 defines a third opening 304a on the first side 212a of the valve portion 136.

As shown in FIGS. 3A and 3B, the second side 212b of the valve portion 136 may be closed and may include one or more grooves or recesses 305. In one embodiment, the second side 212b has two grooves 305 formed diametrically opposed to one another and approximately 90 degrees relative to the first connector flow passage 132a and the second connector flow passage 132b. The grooves 305 may be arcuately shaped.

As shown in FIGS. 3A-3C, the first side 212a of the valve portion 136 may have one or more detents or protrusions 307 extending outwardly from the valve portion 136. In one embodiment, the first side 212a has two detents 307, each diametrically opposed to the other, and each approximately 90 degrees relative to the first connector flow passage 132a and the second connector flow passage 132b.

As described further below, the valve body 120 may have a protrusion 306 extending inwardly from the valve portion 136 into the valve chamber 140. The protrusion 306 is sized and dimensioned to engage a channel 308 (shown in FIGS. 5 and 6) formed in the valve member 124.

Referring now to FIG. 4A, shown therein is a cross-sectional view of the valve body 120 shown in FIG. 3, taken along line 4A-4A. As referenced above, the first connector portion 128a defines the first connector flow passage 132a, and the second connector portion 128b defines the second connector flow passage 132b. Further, the valve portion 136 defines the first opening 300a in fluid communication with the first connector flow passage 132a and the second opening 300b in fluid communication with the second connector flow passage 132b.

As shown in FIG. 4A, the first connector portion 128a may have an outer diameter d₁, the second connector portion 128b may have an outer diameter d₂, the first connector flow passage 132a may have an inner diameter d₃, the second connector flow passage 132b may have an inner diameter d₄, the first opening 300a may have an inner diameter d₅, and the second opening 400b may have an inner diameter d₆. The outer diameter d₁ of the first connector portion 128a may be less than the outer diameter d₂ of the second connector portion 128b. However, in some embodiments, the inner diameter d₅ may be greater than the outer diameter d₂.

The inner diameter d₃ of the first connector flow passage 132a may be equal to the inner diameter d₅ of the first opening 300a and the inner diameter d₆ of the second opening 300b and may be less than the inner diameter d₄ of the second connector flow passage 132b.

The valve body 120 may define a tapered chamber 404 between the second connector flow passage 132b and the second opening 300b, which is in fluid communication with both the second connector flow passage 132b and the second opening 300b. The tapered chamber 404 may have a tapered wall 408 that joins the second connector flow passage 132b to the second opening 300b.

In some implementations, the outer diameter d₂ of the second connector portion 128b is 15 mm; however, in other implementations, the outer diameter d₂ of the second connector portion 128b may be more or less than 15 mm. In some implementations, as shown in FIG. 4B, the inner diameter d₃ may be sized to fit over a 15 mm adapter, whereby the valve body 120 may be connected to a prior art 15 mm adapter rather than used as a substitute for the prior art 15 mm adapter.

As further shown in FIG. 4A, the first connector portion 128a and the first connector flow passage 132a may have a first length l₁, and the second connector portion 128b and the second connector flow passage 132b may have a second length l₂. One or more of the first connector flow passage 132a and the second connector flow passage 132b may be enclosed along an entirety of the first length l₁ and the second length l₂, respectively.

In some implementations, the first connector flow passage 132a is axially aligned with the second connector flow passage 132b; however, in other implementations, the first connector flow passage 132a is not axially aligned with the second connector flow passage 132b.

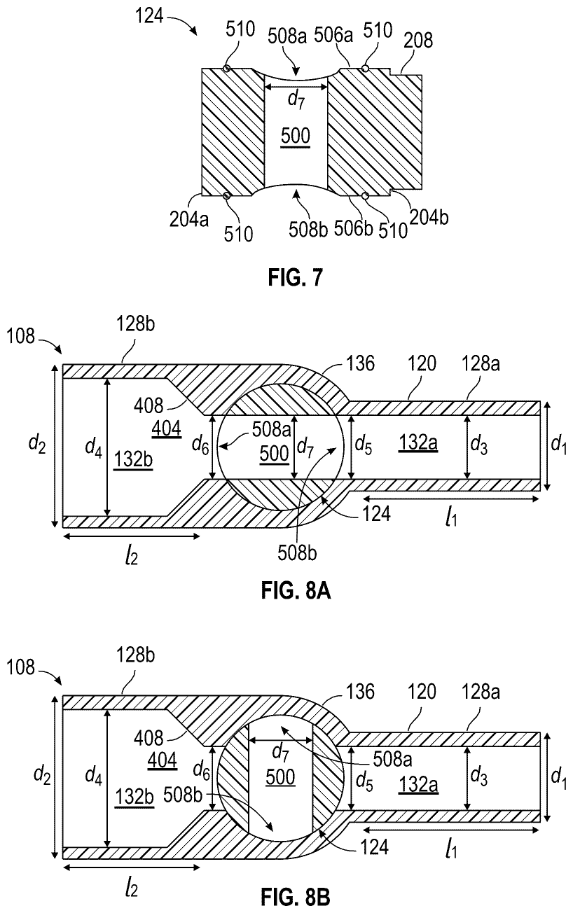

Referring now to FIGS. 5-7, the valve member 124 has a valve flow passage 500 extending therethrough. The valve member 124 may have a cylindrical surface 504 extending between the first end 204a and the second end 204b. The cylindrical surface 504 has a first side 506a and a second side 506b opposite the first side 506a. The valve member 124 defines a first opening 508a on the first side 506a in fluid communication with the valve flow passage 500 and a second opening 508b (shown in FIG. 7) on the second side 506b in fluid communication with the valve flow passage 500.

As referenced above, the valve member 124 may have a channel or groove 308 formed therein sized and dimensioned to engage the protrusion 306 of the valve body 120 shown in FIG. 3. The channel 308 may engage the protrusion 306 such that rotation of the valve member 124 is restricted to a quarter turn. Further, the valve member 124 may be provided with one or more O-rings 510 (hereinafter the “O-rings 510”) surrounding the cylindrical surface 504.

As shown in FIG. 7, the valve flow passage 500 may have an inner diameter d₇. The inner diameter d₇ of the valve flow passage 500 may be equal to one or more of the inner diameter d₃ of the first connector flow passage 132a, the inner diameter d₅ of the first opening 300a, and the inner diameter d₆ of the second opening 300b.

Referring now to FIG. 8A, shown therein is a cross-sectional view of the valve 108 shown in FIG. 2, taken along line 8A-8A, wherein the valve member 124 is positioned in the valve chamber 140 defined by the valve body 120 in the first position (i.e., the open position). As shown in FIG. 8A, when the valve member 124 is positioned in the valve chamber 140 defined by the valve body 120 in the first position (i.e., the open position), the valve flow passage 500 may be aligned with the first connector flow passage 132a and the second connector flow passage 132b, thereby providing fluid communication between the first connector flow passage 132a and the second connector flow passage 132b and allowing fluid flow between the first connector flow passage 132a and the second connector flow passage 132b.

Referring now to FIG. 8B, shown therein is another cross-sectional view of the valve 108 shown in FIG. 2, taken along line 8A-8A, wherein the valve member 124 is positioned in the valve chamber 140 defined by the valve body 120 in the second position (i.e., the closed position). As shown in FIG. 8B, when the valve member 124 is positioned in the valve chamber 140 defined by the valve body 120 in the second position (i.e., the closed position), the valve flow passage 500 may be misaligned with the first connector flow passage 132a and the second connector flow passage 132b, thereby fluidically sealing the first connector flow passage 132a from the second connector flow passage 132b and thereby preventing fluid flow through the valve body 120 between the first connector flow passage 132a and the second connector flow passage 132b.

In use, a user inserts the assembly 100 with the valve member 124 of the valve 108 in the first position (i.e., the open position) into a patient's trachea through the patient's mouth or nose so the assembly 100 extends into the patient's airway. The user inserts a syringe filled with air into the connector portion 160a of the fitting 156 (i.e., the pilot balloon) and depresses a plunger of the syringe to introduce the air into the pilot lumen 154 at the lock end 152b of the pilot line 148. The air travels through the pilot lumen 154 from the lock end 152b to the cuff end 152a and inflates the inflatable cuff 144, thereby forming an airtight seal within the patient's trachea. The user then connects the second connector portion 128b of the valve 108 to a connector of a mechanical ventilator, enabling the ventilator to deliver oxygen to the patient and assist with the patient's breathing.

When the patient is to be disconnected from the mechanical ventilator, the user holds the grip 208 of the valve 108 and rotates the valve member 124 within the valve chamber 140 from the first position (i.e., the open position) to the second position (i.e., the closed position), thereby preventing a transient loss of positive pressure when disconnecting the mechanical ventilator. The user then disconnects the second connector portion 128b of the valve 108 from the connector of the mechanical ventilator and then repeats the process to connect the patient to a different mechanical ventilator, after which the valve member 124 is rotated within the valve chamber 140 from the second position (i.e., the closed position) to the first position (i.e., the open position), so the second mechanical ventilator may deliver oxygen to the patient and assist in the patient's breathing.

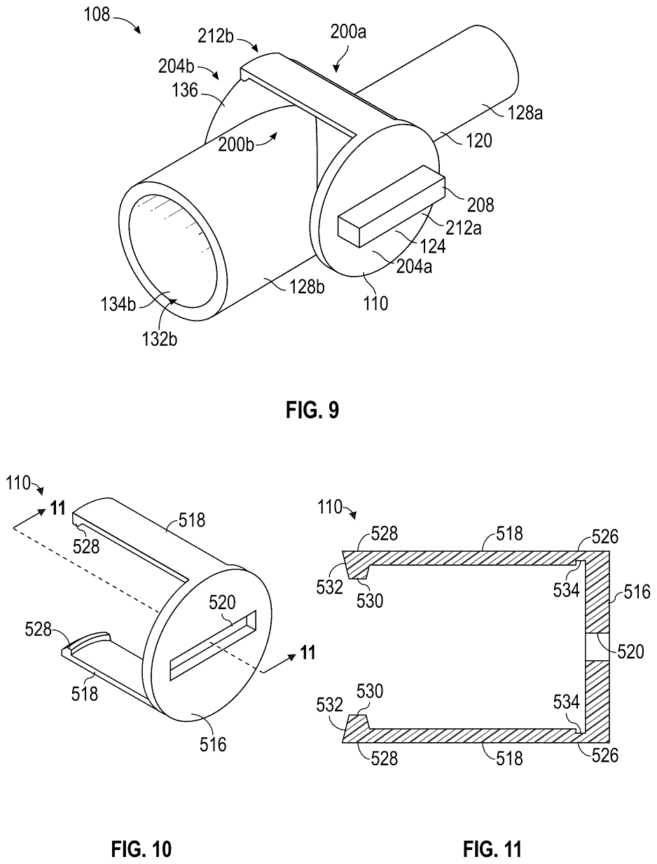

Referring now to FIGS. 9-13, the lock 110 may be employed with the valve 108 to reduce the risk of inadvertent closure of the valve member 124 during use, which could compromise airway patency and patient safety. The lock 110 secures the valve 108 in the open position while allowing for intentional closure when required.

The lock 110 includes a plate portion 516 and two arms 518 extending from the plate portion 516. The plate portion 516 has a slot 520 configured to be matingly received over the grip 208 of the valve member 124 when the valve member 124 is in the open position. The plate portion 516 may be shaped to cover the first side 212a of the valve body 108 and the first end 204a of the valve member 124. In one embodiment, the plate portion 516 may have a circular shape.

The arms 518 extend from the plate portion 516 to be extendible along opposing sides of the valve body 120, and so the arms 518 resiliently extend from the plate portion 516 to be spreadable relative to one another. Each of the arms 518 includes a proximal end 526 and a distal end 528. The distal end 528 is configured to engage the valve body 108 via a detent-and-groove arrangement that provides a snap-fit connection.

In one embodiment, the distal ends 528 of the arms 518 have protrusions 530 that extend inwardly toward one another and are configured to mate with the grooves 305 of the valve body 108. The protrusions 530 may have a beveled surface 532 to facilitate disengagement from the valve body 108, allowing the flexible arms 518 to disengage from the valve body 108 when opposing outward forces are applied to spread the arms 518 and thus disengage the protrusions from the grooves 305 of the valve body 108. The beveled surfaces 532 may be angled 30 to 60 degrees to facilitate disengagement.

The proximal end 526 of each of the arms 518 has a recess 534 that mates with the protrusion 307 of the valve body 108 when the lock 110 is engaged with the valve body 120 and the valve member 124.

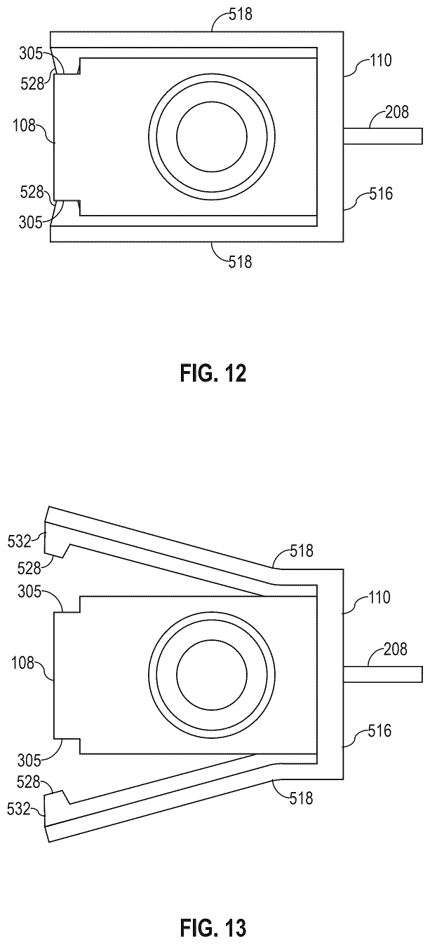

In operation, the lock 110 is positioned over the grip 208 of the valve member 124 by aligning the slot 120 of the plate portion 516 with the grip 208. The arms 516 are pressed against the valve body 120 until the proximal ends 526 and distal ends 528 engage the valve body 120 via the detent-and-groove arrangements, as shown in FIG. 12. This engagement secures the lock 110 in place, preventing the valve member 124 from moving to the closed position.

To remove the lock 110, opposing outward forces are applied to the beveled surfaces 534 of the flexible arms 518, disengaging the protrusions 530 from the recesses 305 as shown in FIG. 13. Once disengaged, the lock 110 may be removed from the valve body 120 and the grip 208 of the valve member 124, allowing the valve member 124 to move freely between the open and closed positions.

The lock 110 may be constructed as a unitary piece and of a suitable material, such as medical-grade plastic, to ensure durability, biocompatibility, and ease of sterilization.

The lock 110 is particularly suited for medical applications requiring secure, reliable control of fluid flow, such as endotracheal tubes. The lock 110 ensures operational safety by preventing inadvertent movement of the valve member 124 while remaining simple to operate and remove. It should be noted, however, that the lock 110 may be used to secure a valve for uses other than medical.

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

What is claimed is:

1. A lock for securing a valve in an open position, the valve comprising a valve body and a valve member with a flow passage extending therethrough, the valve member positioned in a valve chamber of the valve body and movable between an open position, wherein the flow passage of the valve member is aligned with a first connector flow passage and a second connector flow passage to provide fluid communication therebetween, and a closed position, wherein the flow passage of the valve member is misaligned with the first connector flow passage and the second connector flow passage to fluidically seal the first connector flow passage from the second connector flow passage, the valve member having a grip for moving the valve member between the open position and the closed position, the lock comprising:
a plate portion having a slot configured to receive the grip of the valve member when the valve member is in the open position;
a pair of flexible arms extending from the plate portion, the flexible arms configured to extend along opposing sides of the valve body;
a proximal end of the flexible arms configured to engage the valve body; and
a distal end of the flexible arms configured to engage an opposite side of the valve body, the distal ends of the flexible arms being spreadable to disengage from the valve body.

2. The endotracheal tube of claim 1, wherein the slot in the plate portion is dimensioned to prevent movement of the grip when the valve member is in the open position.

3. The endotracheal tube of claim 1, wherein the distal ends of the flexible arms have a beveled surface to facilitate disengagement of the flexible arms from the valve body.

4. The endotracheal tube of claim 3, wherein the beveled distal ends of the flexible arms are angled at 30 to 60 degrees to facilitate disengagement.

5. The endotracheal tube of claim 1, wherein the detent and groove arrangement at the proximal and distal ends of the flexible arms provides a snap-fit connection with the valve body.

6. An endotracheal tube assembly, comprising:
an endotracheal tube having a proximal end and a distal end;
a valve connected to the proximal end of the endotracheal tube, the valve comprising a cylindrical valve body and a valve member positioned in a valve chamber of the valve body, the valve member having a flow passage extending therethrough and being movable between an open position, wherein the flow passage is aligned with a first connector flow passage and a second connector flow passage to provide fluid communication therebetween, and a closed position, wherein the flow passage is misaligned with the first connector flow passage and the second connector flow passage to fluidically seal the first connector flow passage from the second connector flow passage, the valve member further comprising a grip for moving the valve member between the open position and the closed position; and
a lock engaged with the valve to secure the valve member in the open position, the lock comprising:
a plate portion having a slot receive over the grip of the valve member;
a pair of flexible arms extending from the plate portion and extending along opposing sides of the valve body;
a proximal end of the flexible arms engaging the valve body; and
a distal end of the flexible arms engaging an opposite side of the valve body to prevent rotation of the grip relative to the valve body, the distal ends of the flexible arms being spreadable to disengage from the valve body.

7. The endotracheal tube assembly of claim 6, wherein the lock is removably attached to the valve to allow selective locking and unlocking of the valve member.

8. The endotracheal tube assembly of claim 6, wherein the valve body has a pair of grooves formed diametrically opposed to one another, and wherein the distal ends of the flexible arms have protrusions extending inwardly toward one another and matingly received in the grooves of the valve body.

9. The endotracheal tube assembly of claim 8, wherein the distal ends of the flexible arms have a beveled surface to facilitate disengagement of the flexible arms from the valve body.

10. The endotracheal tube of claim 9, wherein the beveled distal ends of the flexible arms are angled at 30 to 60 degrees to facilitate disengagement.

11. The endotracheal tube assembly of claim 6, wherein the proximal ends of the flexible arms are engaged with the valve body.

12. The endotracheal tube assembly of claim 6, wherein the distal end of the endotracheal tube is beveled, and wherein the endotracheal tube has an opening adjacent to the distal end, the opening being in fluid communication with a lumen of the endotracheal tube.

13. The endotracheal tube assembly of claim 6, further comprising an inflatable cuff adjacent to the distal end of the endotracheal tube.

14. The endotracheal tube assembly of claim 13, further comprising a pilot line having a cuff end, a lock end, and a pilot lumen extending between the cuff end and the lock end, the cuff end being coupled to the inflatable cuff and the lock end having a fitting disposed thereon sized and dimensioned to be coupled to a syringe.

15. The endotracheal tube assembly of claim 14, wherein the fitting is a female luer lock fitting.

16. The endotracheal tube assembly of claim 14, further comprising a pilot balloon disposed adjacent to the lock end of the pilot line, the pilot balloon being coupled to the pilot line.

17. A method for maintaining positive airway pressure during the disconnection of a mechanical ventilator from a patient, the method comprising:
providing an endotracheal tube assembly comprising a valve body, a rotatable valve member having a flow passage, a grip, and a removable lock;
positioning the valve member in an open position such that the flow passage is aligned with a first connector flow passage and a second connector flow passage of the valve body to allow fluid communication with the patient's airway;
securing the valve member in the open position by engaging the removable lock with the grip and the valve body, thereby mechanically obstructing rotation of the valve member toward a closed position;
connecting a mechanical ventilator to the second connector flow passage of the valve body;
removing the lock from the valve body and the grip by applying an outward force to spread a pair of flexible arms on the lock;
rotating the valve member via the grip to a closed position to fluidically seal the endotracheal tube and maintain positive pressure within the patient's airway; and
disconnecting the mechanical ventilator from the second connector flow passage while the valve member is in the closed position.

18. The method of claim 17, further comprising the steps of:
connecting a second mechanical ventilator to the second connector flow passage;
rotating the valve member to the open position; and
re-securing the valve member in the open position by re-attaching the removable lock.

19. The method of claim 17, wherein the step of securing the lock comprises snapping a pair of distal protrusions on the flexible arms into corresponding grooves formed on an exterior surface of the valve body.

20. The method of claim 17, wherein the step of securing the lock further comprises aligning a slot in a plate portion of the lock with the grip, wherein the plate portion is configured to cover at least a portion of a side of the valve body.
